(19) United States
(12) Patent Application Publication
(10) Pub. No.: US 2021/0154914 A1
(43) Pub. Date: May 27, 2021

(54) PATIENT SPECIFIC PROTECTION FROM PERIPHERAL RADIATION DURING TREATING CANCER PATIENTS

(71) Applicant: King Abdulaziz University, Jeddah (SA)

(72) Inventors: Abdulraheem KINSARA, Jeddah (SA); Ahmed Sherif EL-GIZAWY, Columbia, MO (US); Essam BANOQITAH, Jeddah (SA)

(73) Assignee: King Abdulaziz University, Jeddah (SA)

(21) Appl. No.: 16/693,487

(22) Filed: Nov. 25, 2019

(51) Int. Cl. B29C 64/118; G06T 17/00; A61B 6/00; A61B 6/10; B29C 64/386

(52) U.S. Cl. CPC B29C 64/118; G06T 17/00; A61B 6/584; B33Y 30/00; B29C 64/386; G06T 2207/10081; A61B 6/107

(57) ABSTRACT

A method for making radiation shielding or a hollow coupon that can be filled with a material that blocks or absorbs radiation. The invention also encompasses radiation shielding made by this method and a method of using radiation shielding during clinical irradiation procedure.

### BACKGROUND OF THE INVENTION

Field of the Invention. The invention pertains to the field of medical radiology and manufacture of radiation shielding using 3D-printing or additive manufacturing.

Description of Related Art. A total of 1,660,290 new cancer cases and 580,350 cancer deaths occurred in the United States in 2013; see Siegel, R., Naishadham, D. and Jemal, A., 2013. Cancer statistics, 2013. CA: a cancer journal for clinicians, 63(1), pp. 11-30. Radiation therapy is often used for cancer treatment.

Current radiation therapy using a linear accelerator (LINAC) is an effective way to kill all kinds of tumors. A device called multileaf collimator (MLC) is used for shaping the x-ray beam to conform to the patient's tumor. A MLC consists of a certain number of individual “leaves” of a high atomic numbered material, usually tungsten, that move independently in and out so that the radiation beam conforms to a tumor's shape. However, current designs of MLCs produces radiation leakage and scatter in an amount of about 2-10% of the maximum dose given to the patient; see Kinsara A, El-Gizawy A S, Banoqitah E, Ma X (2016), Review of Leakage from a Linear Accelerator and Its Side Effects on Cancer Patients. J Nucl Med Radiat Ther 7: 288. doi:10.4172/2155-9619.1000288; Kinsara, A., El-Gizawy, A. S., Ma, X., Characterization of Attenuating Properties of Novel Composite Radiation Shields. Journal of Nuclear Medicine & Radiation Therapy, 2016, 7:6; Van de Walle J, Martens C, Reynaert N, Palmans H, Coghe MARC, et al. (2003) Monte Carlo model of the Elekta SLiplus accelerator: validation of a new MLC component module in BEAM for a 6 MV beam. Physics in medicine and biology 48: 371; Klüter S, Sroka-Perez G, Schubert K, Debus J (2010), Leakage of the Siemens 160 MLC multileaf collimator on a dual energy linear accelerator. Physics in Medicine and Biology 56: N29; and Tello, Victor M., Medical Linear Accelerators and how they work, text available at hypertext transfer protocol://hpschapters.org/florida/13PPT.pdf (last accessed Mar. 22, 2018).

In case of a dose around 7000cGy to treat a cancer patient, healthy organs and tissues are exposed to a range of radiation from 140cGy up to 665cGy where a dose as low as 10cGy may cause damaging effects to healthy tissues.

Conventional radiation shielding is often one-size-fits-all. However, patients needing radiation treatment have different anatomies and different radiation treatments may involve different distances and positioning of medical equipment. Thus, use of one-size-fits all radiation shielding during treatment can result in high levels of leaked or scattered radiation into non-target tissues.

Additive manufacturing of 3D printing has been used to produce radiation shielding. However, such radiation shielding has many drawbacks. Many 3D printing method do not take into account patient anatomy and engineering factors necessary to produce a safe and efficacious radiation shield. Moreover, they print or additively manufacture using heavy, radiopaque 3D printing materials such as a thermoplastic mixed with heavy metal powder; U.S. 2015/0257313 A1, U.S. 2015/0048209A1, or CN206535012. Such printing requires control and handling of these heavy metal-containing “inks”. Moreover, once a radiation shield is printed with such a heavy, metal-containing material it is not feasible to further modify its shielding properties.

In view of the drawbacks of conventional radiation shielding and methods of manufacturing it, the inventors sought to develop an improved and more flexible method for designing and manufacturing radiation shielding to meet the urgent need for precise targeting of radiation to sites of cancer cells and to reduce radiation treatment side effects caused by misdirected, scattered, or leaked radiation during treatment.

### BRIEF SUMMARY OF THE INVENTION

The methods disclosed herein provide a superior, patient- and procedure-customized radiation shielding that increases the safety and accuracy of external beam radiation therapy (“EBRT”) by facilitating radiation of a target site and by blocking radiation scattering to normal, non-target tissues. It reduces the side effects of external bean radiation treatments for cancer patients and provides an economical way to customize radiation shielding to a patient's anatomy, the anatomy of a tumor or other target site, and to the constraints of a particular kind of EBRT. Moreover, the customized radiation shielding of the invention can decrease radiation leakage and scattering by 50-70%. While the shielding is advantageously designed to protect patients undergoing EBRT it may also be used for any other procedure where shielding is required including for diagnostic procedures such as X-rays, CT scans and other diagnostic or medical procedures involving radiation.

The customized shielding of the invention can be designed to protect sensitive parts in the body such as heart, lung, or thyroid or tissues, developing or growing tissues such as those in children or fetal tissues, and sites containing implantable devices like pace makers from exposure to radiation. It also reduces the risk a secondary malignancy developing in normal tissue when exposed to scattered radiation during a cancer treatment.

Generally, after consultation with medical specialists such as doctors, radiologist and medical physicists, a radiation shield design is selected based on data from a scan of a prospective patient's anatomy and then virtually evaluated and further modified as necessary. The shield may be designed as a single piece or multiple pieces depending on the specific needs of a patient and on the nature of the radiological procedure. Once the virtual or actual analysis of the shield design is complete, a prototype hollow shell or coupon is 3D printed, usually from a resin such as a polycarbonate that does not contain a heavy metal component. The 3D printed prototype coupon or shell is then filled or loaded with a radiation blocking material such as bismuth, lead, or tungsten to produce prototype radiation shielding which can be tested on a phantom or dummy patient equipped with radiation sensors. Further modifications to the design of the hollow coupon or shell or to the prototype may be made based on the evaluation of the prototype. After virtual and prototype analysis is completed, a final hollow coupon or shell is 3D printed, filled with an amount of an appropriate radiation blocking material, and prepared for use by the patient.

Many embodiments of the invention involve one or more of the following (i) evaluation of an attenuation rate of coupons with different structural designs or different engineering constraints (where each design represents a different scenario that is separately evaluated), (ii) design and development of a shield that takes into account the patient's anatomy and the anatomy of a treatment site such as that of a tumor using a digital technology approach (“DTA”), (iii) virtual evaluation of suitability and dimensional accuracy of the developed shields using 3D models created or derived from patient scanning data, (iv) finite element analysis of safety and engineering factors of a shield model, and (v) verification of the effectiveness of the designed and developed shield using 3D printed full-scale prototypes which are tested on phantoms equipped with radiation sensors.

One embodiment of the method of the invention involves making radiation shielding for a particular patient and particular radiological procedure by (a) scanning a body or body part of a subject to produce scanning data that describes a target site to be treated with radiation and non-target sites to be protected; (b) inputting scanning data into a CAD program to produce a 3D CAD model of a hollow shell or coupon containing one or more cavities that can accommodate radiation blocking material and that is shaped so that when placed on the body of the subject exposes a target site to be irradiated and shields one or more non-target sites from radiation; (c) digitally assessing the 3D CAD model to determine distribution of stresses and deformations in the model shielding when filled with a radiation shielding material and selecting a 3D CAD model that is assessed to have a strength suitable for clinical use; (c) 3D-printing or additively manufacturing (“AM”) a prototype radiation shielding from the selected 3D CAD model; and, optionally, (e) testing the 3D-printed or additively manufactured prototype on a phantom subject equipped with one or more radiation sensors. The scan used in this method may be a CT/X-ray scan, MRI scan, or other medical scan that localizes a target site or non-target sites or tissues.

A CT scan, also known as computed tomography scan, makes use of computer-processed combinations of many X-ray measurements taken from different angles to produce cross-sectional (tomographic) images (virtual “slices”) of specific areas of a scanned object, allowing the user to see inside the object without cutting. Other terms include computed axial tomography (CAT scan) and computer aided tomography. Digital geometry processing is used to further generate a three-dimensional volume of the inside of the object from a large series of two-dimensional radiographic images taken around a single axis of rotation. Medical imaging is the most common application of X-ray CT. Its cross-sectional images are used for diagnostic and therapeutic purposes in various medical disciplines.

Magnetic resonance imaging is a medical imaging technique used in radiology to form pictures of the anatomy and the physiological processes of the body in both health and disease. MRI scanners use strong magnetic fields, electric field gradients, and radio waves to generate images of the organs in the body. MM does not involve X-rays and the use of ionizing radiation, which distinguishes it from CT or CAT scans. Magnetic resonance imaging is a medical application of nuclear magnetic resonance (NMR).

The scan may be of the whole body or a portion of the body and will usually include the target site (e.g., a tumor mass) and anatomical sites close to it most likely to be exposed to scattered or leaked radiation during a radiation treatment. Thus, another aspect of this embodiment involves scanning a subject's neck, head or portion thereof; a subject's torso or portion thereof; a subject's thorax or portion thereof; a subject's abdomen or portion thereof; a subject's arm or leg or portion thereof; scanning a position of tumor mass or tumor site to be exposed to radiation; or locating a position of one or more organs (e.g., brain, spinal cord, eyes, ears, heart, nose, bronchi, lung, stomach, intestine, liver, spleen, skin), glands (e.g., hypothalamus, pineal, pituitary, thyroid, parathyroid, thymus, adrenal, kidney, pancreas, ovaries, uterus, mammary glands, testes, penis, prostate) or other tissues to be protected from radiation.

In another related embodiment, the medical scan will locate a position of an embryo or fetus to be protected from radiation in a pregnant woman or positions of germ cells or other reproductive tissues in female or male subjects.

The scanning may also locate a position of one or more pacemakers, prosthetics, or implanted devices to be shielded from radiation.

In the above embodiments, the medical scanning data will be used to produce a 3D model, such as a 3D-CAD model of the target site and/or non-target sites to be protected from misdirected, leaked, or scattered radiation. Data from medical scans is transmitted or converted into a form suitable for 3D-CAD modeling. For example, a DICOM file from a CT scan may be converted to an NRRD file using programs known in the art such as 3D Slicer. As disclosed herein programs like Materialise Mimics to construct 3D models.

The type of scan and extent and required features for a 3D model are usually specified by medical professionals such as oncologists, medical physicists, and radiologists. Planning for a radiation treatment of a particular subject will usually include the identification of the target site which will be exposed to and treated with radiation, the type and dosage of radiation, the dimensions of a margin around a target site, identification of non-target sites including tissues, organs or glands to be protected and the degree of protection of these tissues from exposure to radiation. Each of these factors may be taken into account to design a model of the shielding (or a model of a hollow coupon or shell that will hold the shielding).

A finite element analysis using the 3D model is performed based on engineering parameters to evaluate the durability and stiffness of a designed shield. Constraints that may be taken into account include bodily areas covered by shielding, the kinds and amounts of radiation-blocking materials, the thickness of the shielding or volume of fillable cavity in a radiation-shielding shell or coupon, the kinds of materials used to make the shell (e.g., polycarbonate, other resins, and whether the shell will contain radiation blocking materials), the weight of the shell, the durability, strength and fracture resistance of the shell and the shell when filled with a radiation blocking material, the cost and time required to produce the shell and shell filled with a radiation-blocking material. Representative radiation blocking materials include bismuth, lead or tungsten or mixtures of these.

A 3D model and analysis of a 3D model can involve modeling an integral radiation-shield or modeling a shield that has two or more parts (or subportions), such as a left and right part or an upper and lower part, or a top and bottom part which are later assembled to form a template containing a space for loading of a radiation blocking material. A coupon, shell or part or subportion may be symmetrical or non-symmetrical. A shell may contain two or more parts that when assembled or adhered together form a hollow compartment which can be loaded with a radiation blocking material. In some embodiments one or more parts of a shell is filled with a radiation blocking material prior to assembly with its component part or parts. A hollow coupon or shell (or a filled coupon or shell) may have arms, legs, or other supports that permit it to rest on a solid surface or that attach it to another support, for example, as shown in FIG. 5.

In some embodiments the 3D model and subsequently printed coupon or shell will contain an exposure aperture (or shieldless area, hole, groove, finger, or other extension) which is later oriented above or next to a target site to provide an unshielded portion through which external radiation may pass to irradiate a target site. The exposure aperture may be designed to cover target sites with different shapes or contours as well as a margin of tissue around the target site, for example, the exposure aperture may include space for a 0.125, 0.25. 0.5, 0.75, 1.00, 1.25, 1.5, 1.75 or 2.0 cm margin around the location of a tumor in a cancer patient which is also irradiated along with (or as part of) the target site. In other embodiments, the aperture will be precisely fitted to the size of the target site, such as the size of a well-defined tumor mass so as to avoid irradiation of tissues surrounding the target site.

The thickness of the coupon or shell will be selected to provide sufficient support for the weight of the radiation-blocking material to be incorporated or loaded into the coupon or shell. Some embodiments will have thicknesses ranging from 0.02, 0.05, 0.075, 0.1, 0.2, 0.5, 0.75, 1.0, 1.2, 1.5, 2.0, or >2.0 cm (or any intermediate value within this range) though coupon or shell thickness may fall outside this range for some types of shielding.

The volume of the hollow coupon or shell, which provides room for a corresponding amount of radiation-shielding material, will be selected based on the type of radiological procedure and the degree of shielding required. For example, it may be selected to block 5, 10, 15, 20, 25, 30, 35, 40, 45, 50, 55, 60, 65, 70, 75, 80, 85, 90, 95 or <100% of the misdirected or scattered radiation from irradiation of a target site.

Some representative thickness radiation-shielding materials are 0.1, 0.2, 0.5, 1.0, 1.2, 1.5, 2.0, 5.0, 10.0, 12.0 15.0 and 20.0 cm (or any intermediate value with this range), though thickness may fall outside this range for some radiological procedures. These values refer to the thicknesses of the radiation blocking materials inside a coupon or shell. In embodiments, where a coupon or shell may incorporate a radiation blocking material, the thickness of the coupon or shell in addition to a filling or volume of radiation blocking material may fall within these ranges. Coupon volume will correspond to the amount of radiation blocking material needed to be loaded into the coupon. Coupon volume and amount of shielding (when the coupon is loaded with a radiation blocking material) may vary over the area of the shielding, thus, shielding may be thicker in some areas than in others.

The 3D model is digitally assessed to determine distribution of stresses and deformations imposed by a weight of the radiation shielding when filled with a radiation shielding material. The assessment may involve a Von Mises stress analysis. When the 3D model has two or more portions, which may be symmetrical or non-symmetrical, both parts may be digitally assessed to determine stress distribution.

Once stress and other engineering factors are assessed, adjusted or adapted to produce shielding for a particular patient and radiological procedure, the model may be virtually tested for its ability to block radiation to a virtual phantom subject.

Additionally or alternatively, a model coupon or shell may be 3D-printed or additively manufactured, filled with a radiation blocking material to produce one or more prototypes. The prototypes may then be tested for their capacity to allow radiation to teach a target site, to block radiation to nontarget sites, calculate relative ability to block In some embodiments, a prototype will be tested on an actual, not virtual, phantom subject which mimics a contour or position of a portion of the subject's body containing the target site to be irradiated and non-target sites to be protected from radiation. The phantom subject is equipped with one or more radiation sensors to determine an amount of radiation to which target and/or non-target sites are exposed. Based on the results of the virtual and/or actual phantom testing, the 3D model may be further modified to minimize exposure of non-target sites to radiation during a radiological treatment or to enhance actual or relative (to non-target sites) amounts of radiation delivered to a target site. Sensors on a phantom subject may be gel dosimeters.

Results from the virtual and/or actual phantom testing are used to validate the 3D model being tested or as a starting point for additional modifications to a coupon, shell, or filled coupon or shell, model. Once a final model has been selected, the coupon, shell or components thereof are 3D printed or additively manufactured, filled with a selected amount of a selected radiation blocking material, assembled if necessary, and then fitted to a subject who will be undergoing radiological treatment. In some embodiments, a model of a coupon or shell may be directly 3D printed or additively manufactured, filled with a radiation blocking material, and fitted to, or used by a patient without prototyping and further testing.

Another embodiment of the invention is a 3D-printed or additively-manufactured radiation shielding that is made by the methods disclosed herein. The shielding may cover or protect one or more tissues, organs, glands or other non-target sites such as those disclosed herein.

The invention also contemplates and pertains to methods for treating a subject with radiation comprising covering non-target sites of the subject's body with the radiation shielding produced by the methods described herein. A treatment method may treat cancers, neoplasms, tumors, proliferative conditions or other conditions that are treatable with external beam radiation, including those disclosed herein.

### BRIEF DESCRIPTION OF THE SEVERAL VIEWS OF THE DRAWING(S)

A more complete appreciation of the invention and many of the attendant advantages thereof will be readily obtained as the same becomes better understood by reference to the following detailed description when considered in connection with the accompanying drawings, wherein:

FIG. 1. Medical Linear Accelerator (LINAC) used for Radiation Therapy.

FIG. 2. Digital Technology Approach (DTA).

FIGS. 3A-3D provide several views of a patient's CT scan and the detailed anatomy of the trunk. The specific treatment plan for the patient offers the clinical constrains for the engineering design, analysis, and manufacturing.

FIG. 4. 3D model generated from CT scan.

FIG. 5. 3D model of shield designed to match patient's anatomy.

FIG. 6. Von Mises stress distribution due to the device own weight.

FIG. 7. Deformation (distortion) distribution due to the device own weight.

FIG. 8. Schematics of Fusion Deposition Modelling (3D Printing) process used in construction of the device.

FIG. 9A The developed split shield design is displayed.

FIG. 9B. The assembled shield is shown.

FIG. 10A Set up for testing the developed shield under LINAC machine.

FIG. 10B Set up for testing the developed shield under LINAC machine.

FIG. 11. Flowchart of a method for making one embodiment of the invention. Reference numbers 100, 110, 120, 130, 140, 150, 160 and 170 describe steps in the method.

FIG. 12A. Fused deposition modelling (FDM) 3-D printing process.

FIG. 12B. Modular configuration of shield design.

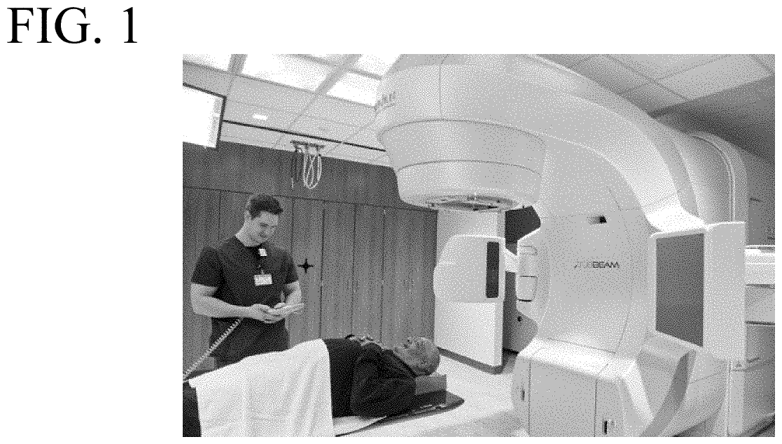

### DETAILED DESCRIPTION OF THE INVENTION

The invention provides a new way to custom design radiation shielding for a particular patient and radiological procedure to reduce exposure of the patient to misdirected or scattered radiation. Some aspects of this technology are known under the name TECHSHIELD.

A medical scan is performed on the prospective patient and a 3D-CAD model of the patient made. The 3D model of the patient serves as a template for designing a model of radiation shielding conforming to the patient's anatomy and protecting tissues and organs outside of the target site for radiation treatment.

Relevant factors for engineering the radiation shielding are evaluated using the 3D-CAD model of the shielding using a finite element analysis. Such analysis helps identify models that have the strength to protect the patient without collapsing and hurting the patient and without structural distortion of their radiation blocking ability under a heavy load of radiation blocking material when loaded into the hollow 3D printed coupon or shell. Different virtual models of the shielding may be tested to evaluate their abilities to safely protect the patient from scattered or misdirected radiation during a radiological procedure.

Once a suitable model is designed and selected, a 3D printed hollow coupon or shell is produced, loaded with a radiation blocking material prior to clinical use. In some embodiments, a prototype 3D printed coupon or shell is loaded with a radiation blocking material and tested on a phantom equipped with radiation sensors. Data from the testing of the prototype is used to further refine the 3D model.

Radiation therapy. In radiation therapy (also called radiotherapy), invisible high-energy rays or beams of subatomic particles are used to damage cancer cells and can stop them from growing and dividing. Therapeutic radiation inhibits cancer cell growth and proliferation and ultimately can kill the cancer cells treated. Radiation therapy may aim to shrink a tumor or cure it. Such therapies may be conducted after administration of other therapeutic agents such as anticancer drugs or hormones (neoadjuvant therapies), in conjunction with other therapies (adjuvant therapies), or as part of a palliative therapy.

The invention may be used in various modes of external-beam radiation therapy including LINAC-based therapies, 3-dimensional conformational radiation therapy (“IMRT”), image-guided radiation therapy (“IGRT”), tomotherapy, sterotactic body radiation therapy (“SRBT”), proton therapy, neutron therapy, heavy ion therapy (e.g., boron, carbon, neon ions), boron neutron capture therapy (“BNCT”), gadolinium neutron capture therapy, and radiotherapies using X-rays, gamma rays, or other charged particle beams. In addition to EBRT, the shield may be used in diagnostic procedures in which non-target tissues in a patient are at risk of exposure to radiation including CT scans and X-ray procedures or where shielding against scattered radiation improves image quality or a scan or X-ray image.

One mode of radiation therapy uses a linear accelerator (“LINAC”), a device that accelerates radioactive particles and beams them into body regions affected by a malignancy. A linear accelerator can be used to treat many different kinds of malignancies in different parts of the body. In many applications, it delivers high-energy x-rays or electrons to the region of a patient's tumor and delivers therapeutic radiation in the range of 4 to 25 million volts, as either intense radiation or high-energy electron beams—most commonly, ⁶⁰Co, delivering 2-10 Gy/min (200-1,000 rads/min) at the center of an internal malignancy.

A potential risk and side-effect of external beam radiation therapy is that it creates misdirected, leakage and scatter radiation into non-cancerous tissues and can damage these tissues or even cause secondary malignancies.

The customized shielding provided by the invention reduces exposure to these kinds of misdirected, leaked or scattered radiation thus reducing, or reducing the risk of, one or more acute, later or cumulative side-effects of radiation treatment. These include acute side-effects such as nausea, vomiting and diarrhea, damage to epithelial surfaces, sores such as mouth, throat and stomach sores, intestinal discomfort, swelling or infertility; or later side-effects such as epilation, dryness, lymphedema, secondary malignancies, heart disease, cognitive decline, or radiation enteropathy. Cumulative effects such as risk of infertility, damage to reproductive system, damage to germ cells, damage to fertilized eggs, embryos or fetuses, damage to the thyroid, pituitary, thymus, and salivary glands, lymphoid organs, bone marrow, testes, ovaries, and other radiosensitive tissues such as epithelial cell linings, and growing bone or cartilage.

Cancers. The invention may be used to treat cancers and other conditions treatable using external-beam radiation therapy in which normal or non-cancerous tissues or organs of a patient are at risk of exposure to misdirected, leaked or scattered radiation. Generally, leukemias and other disseminated or metastasized cancers are not curable with radiation therapy, because they are disseminated through the body. Lymphoma may be radically curable if it is localized to one area of the body. Similarly, many of the common, moderately radioresponsive tumors are routinely treated with curative doses of radiation therapy if they are at an early stage. For example: non-melanoma skin cancer, head and neck cancer, breast cancer, non-small cell lung cancer, cervical cancer, anal cancer, and prostate cancer. Common kinds of cancers include bladder cancer Radiation therapy may also be used to treat non-malignant conditions, such as trigeminal neuralgia, acoustic neuromas, severe thyroid eye disease, pterygium, pigmented villonodular synovitis, prevention of keloid scar growth, vascular restenosis, or heterotopic ossification. Radiation therapy may also be used to treat early stage Dupuytren's disease and Ledderhose disease. Cells of self-renewing tissues, such as those in the crypts of the intestine, are the most radiosensitive. Cells that divide regularly but mature between divisions, such as spermatogonia and spermatocytes, are somewhat less radiosensitive. Long-lived cells that usually do not divide unless there is a suitable stimulus, such as liver, kidney, and thyroid cells, are even less radiosensitive. Least radiosensitive are cells that have lost the ability to divide, such as neurons. Skin and other organs with epithelial cell lining (cornea, oral cavity, esophagus, rectum, bladder, vagina, uterine cervix; to block radiation to one or more moderately radiosensitive organs or tissues including the optic lens, stomach, growing cartilage, fine vasculature, or growing bone; or to block, prevent damage or side-effects to one or more moderately low sensitive organs or tissues mature cartilage or bones, salivary glands, respiratory organs, kidneys, liver, pancreas, thyroid, adrenal and pituitary glands; or to block, prevent damage or side-effects to one or more lowly sensitive organs or tissues including muscle, brain, and spinal cord and other nervous system tissues including those of the enteric nervous system.

Scans. A computed tomography (CT) scan is taken of one or more subjects. This scan combines a series of X-ray images taken from different angles and uses computer processing to create cross-sectional images, or slices, of the tissues inside the body. In some embodiments data from magnetic resonance imaging (“MM”), positron emission tomography (“PET”), or ultrasound scans may be used to model a site to be treated for radiation, or used in combination with data from a CT scan. Other scanning methods known in the art may also be used, such as those described by and incorporated by reference to Shin, U.S. Pat. No. 8,527,244.

One skilled in the art may select an appropriate software tool for finite element methods used for drug evaluation. One embodiment of the invention uses medical image processing software such as Materialize Mimics for assessing the geometric properties of the patients' and tumors' outlines. Materialise Mimics is an image processing software for 3D design and modeling, developed by Materialise NV, a Belgian company specialized in additive manufacturing software and technology for medical, dental and additive manufacturing industries. Further description of Materialized Mimics software and methods of use is incorporated by reference to hypertext transfer protocol://www.materialise.com/en/products-and-services. Embodiments may use a simulation software such as “MicroShield” to evaluate virtually the effectiveness of earlier simple coupons filled with heavy materials. Analytical models can also be used to virtually predict the effectiveness of the tested coupons such as those described by Kinsara, A., et al., Characterization of Attenuating Properties of Novel Composite Radiation Shields. Journal of Nuclear Medicine & Radiation Therapy, 2016 (incorporated by reference). Materialise Mimics (commercial software) may be used to convert the CT scans into 3D model (CT segmentation techniques) for design, analysis and manufacturing.

Segmentation techniques are applied to the slices and convert the cross-sectional images into a 3D CAD model of tissues in and around a radiation target site (e.g., a tumor or other mass), a margin around the target site, and tissues and organs (e.g., heart, lung, gland, fetus, implant, etc.) to be protected from scattered, leaked or other misdirected radiation.

Medical physicists and physicians who plan and conduct the radiation therapy are involved in establishing the clinical design constraints of the shield. An evaluation of the attenuation rates of coupons produced using different design parameters when filled with different kinds of radiation blocking materials.

Design parameters may include one or more of the following bodily area or organ to be covered or protected, type of cancer or other condition to be treated with radiation, age, sex and other bodily parameters of a patient to be treated, stage, radio sensitivity and physical location of a tissue to be irradiated, dimensions of margin around tissue to be treated, type of radiation absorbing material, type or particle or wavelength of radiation to be blocked, amount of radiation to be blocked, thickness of shield, thickness of shell, shell materials, shell and filled shell weight, patient positioning and spatial positioning of a shield on a patient during treatment, production time for modeling and additively manufacturing shield, and production cost.

Based initial clinical and engineering design considerations, a 3D CAD model of a shield, in single or multiple pieces is constructed. Generally, the model will describe a shell-like structure or coupon containing one or more cavities for accommodating radiation blocking material such as heavy metal particles.

A model of the coupon when filled with a radiation blocking material is tested to evaluate its ability to attenuate radiation over non-target sites and/or permit radiation to be received at a target site.

A finite element analysis model is created to evaluate the durability and stiffness of the designed shield. The finite element analysis used in the development of the patient-specific shields is an existing method for structural analysis (data analyses technique). Finite element analysis (FEA) is used in the development process to check the reliability and the safety of the shield for carrying the required load without failure (collapsing and hurting the patient) or even distortion (changing the geometric characteristics of the shield), that might interfere with the function and effectiveness of the shield. However, the inventors apply FEA to additive manufactured (3D printed), complex structure with anisotropic properties (variation of properties along the three principle axes). This unique approach modifies the standard FEA to adjust to the variation of properties without losing the accuracy of the results.

Data analysis for development of the patient-specific shields. These include determination of attenuating properties of potential filling heavy metal particles and the design factors controlling the shielding effects of the developed device. A statistical design using the following parameters or variables was used the width of the internal cavity (filler material thickness), the plastic shell material thickness, and the type and size of the filling heavy metal particles, as experimental design variables.

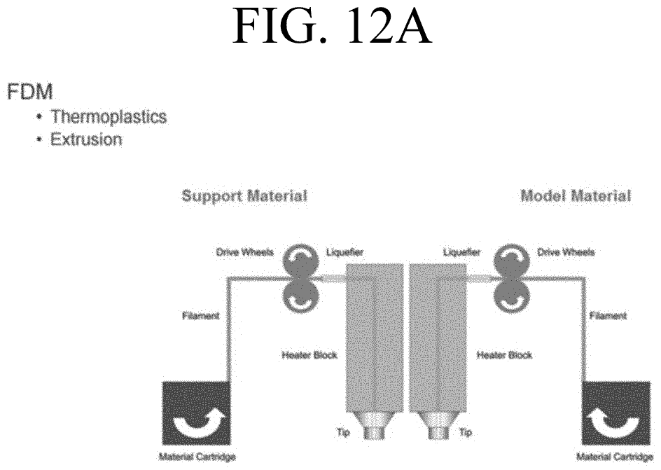

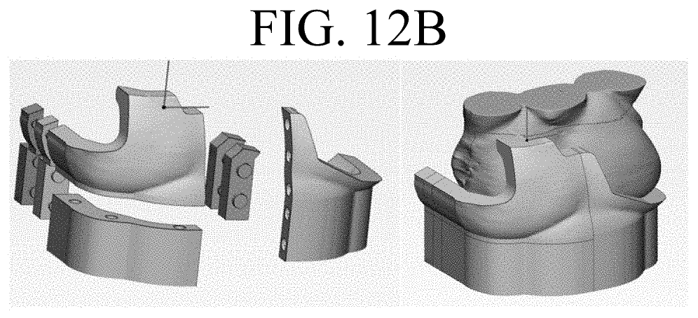

Shell/Coupon. The shell or coupon is preferably made out of a thermoplastic material polycarbonate (PC) or other suitable materials. The shell is typically constructed by an additive manufacturing (AM) technique using the Fused Deposition Modeling (FDM) method on STRATASYS's FORTUS 400 mc system. This is a preferred method which can be used to print shields and coupons. The basic principles of FDM are described by FIG. 12A. To facilitate production using FDM the inventors have designed a shield with an added modular configuration; FIG. 12B. This modular configuration divides the shield into separate components that can later be assembled. This provides flexibility and permits adjustments for different kinds of patients and reduces fabrication costs.

Polycarbonates (“PC”) are a group of thermoplastic polymers containing carbonate groups in their chemical structures. Many polycarbonates strong, tough materials and some grades are optically transparent. They are easily worked, molded, and thermoformed. Because of these properties, polycarbonates find many applications. Polycarbonates made from polycarbonate may contain the precursor monomer bisphenol A (BPA) and contain the repeating unit:

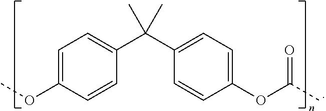

Polycarbonate-based 3D printing materials are used to 3D-print the coupon or shells of the invention. Polycarbonates exhibit good strength and impact resistance, but can be subject to stress cracking. It may also shrink and warp during 3D printing. The method of the invention solves many problems associated with use of polycarbonates for 3D printing radiation shielding for clinical use.

Alternatively, in some embodiments non-polycarbonate thermoplastic 3D printing materials or polycarbonate mixtures may be used to design prototype shells or coupons or in a final shell or coupon to be filled for shielding. Thermoplastics used for 3D printing include polylactic acid, poly(acrylonitrile butadiene styrene), polystyrene, nylon, high density polyethylene, polycarbonate, polyvinyl alcohol, and polyethylene terephthalate or mixtures thereof. In some embodiments, a polymer material for 3D printing has a first polymer and a second polymer. The first polymer and the second polymer may be crosslinked by a photo-crosslink forming a polymer network. The first polymer and the second polymer may be independently selected from polylactic acid, poly(acrylonitrile butadiene styrene), polystyrene, nylon, high density polyethylene, polycarbonate, polyvinyl alcohol, and polyethylene terephthalate. First and second polymers also include thermoplastic polymers such as styrenic block copolymers (thermoplastic elastomers, TPE-s), thermoplastic olefins (TPE-o), elastomeric alloys (TPE-v or TPV), thermoplastic polyurethanes (TPU), thermoplastic copolyesters, and thermoplastic polyamides.

Radiation-blocking materials. A radiation-blocking material used to fill a 3D-printed coupon may be in various forms, such as powder, granular, or pellets, or may be in admixture with a liquid, viscous or curable material such as a resin, such as polyurethane, that can be injected, pumped or otherwise loaded into a hollow shell or coupon. A filler composition may contain a polyolefin elastomer, a polyolefin copolymer a polyolefin ter-polymer or combinations thereof in admixture with a metal-containing or metal-compound-containing filler; see U.S. 2014/0117288 (incorporated by reference). Metal compounds include metal oxides and glasses, such as lead oxide or lead-based glasses. Some representative materials include heavy metal particle fillings such as lead, bismuth, tungsten, tin, antimony, and composites thereof.

Effective blocking materials include heavy metal materials such as lead, bismuth, and tungsten. These high density metals exhibit both cost effectiveness and excellent attenuation rates. All the proposed blocking or filling materials are available in powder, granular or pellet forms, which can be easily filled into the shield cavity through well-designed inlets on the shield. The location and number of inlets will be designed according to the specific patient case.

Evaluation of Prototype Shielding. Shielding provided by prototype coupons is evaluated using a LINAC machine used for radiation therapy of cancer patients. LINAC can deliver X-ray beams in the range of 4 MeV to 20 MeV. A Cobalt-60 source that emits gamma rays of 1.17 MeV and 1.33 MeV is used in part of the experimental investigation to simulate a radiation beam using gamma rays during cancer treatment.

The blocking percentage is identified as the important response of the experimental investigation and is used for evaluation of the effectiveness of the developed shielding device. It is calculated from the following equation:

Blocking Percentage=[1−(Penetrated Photon Counts/Total Photon Counts)]×100

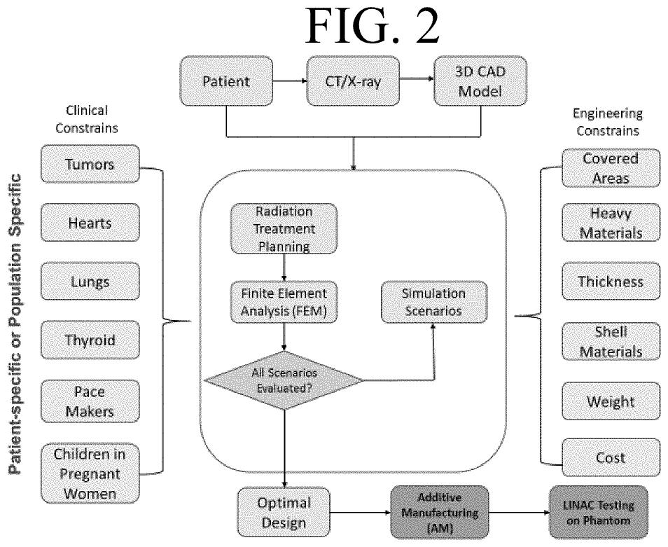

This is larger the better case since the larger the blocking percentage means the better attenuation property of the device. The investigation results are used for predicting the design variables that give the most effective shielding under the given engineering and clinical constraints (see FIG. 2).

Suitability and dimensional accuracy of the developed shield are evaluated virtually using the patient's 3D model.

Additive Manufacturing (AM), also called 3D Printing, digitally develops products in a layer wise fashion that correspond to the build files created from the CAD model. AM technologies are superior to conventional fabrication techniques for producing parts with complexity and cost effective for individual customization.

The 3D model of the shield is manufactured by AM techniques and filled with metallic particles (lead, bismuth, tungsten, etc.).

Verification the effectiveness of a manufactured shield involves testing of full-scale prototype shields utilizing the introduced novel digital technology approach (DTA) presented in FIG. 2.

These prototypes may then be tested using phantoms that have similar configuration of patients. Phantoms are equipped with several sensors to measure the effectiveness of a design for reducing peripheral radiation dose during treatment of a patient with radiation. Phantoms may be equipped with dosimetry sensors including those used for determining three-dimensional dose distributions in gel dosimetry; incorporated by reference to Baldock, C, et al. (2010). Polymer gel dosimetry. Physics in Medicine and Biology. 55 (5): R1. doi:10.1088/0031-9155/55/5/r01. In other embodiments, radiation shielding is made without prototyping and further testing by 3D printing or additively manufacturing a coupon or shell, filling it with a radiation blocking material, and sealing, covering or otherwise preparing the shielding to be fitted or used by a patient undergoing a radiological procedure such as EBRT. In some embodiments, the shielding may be coated, painted or otherwise covered with paint or an external coating such as a plastic coating or paper, cloth or fibrous covering, or fitted with handles or grasps to facilitate fitting it to a patient or its clinical use. Preferably, coatings or films are not applied around the shell or coupon wall closest to the radiation source to further focus or block radiation.

In some embodiments, the walls of the shell or coupon, which are adjacent to or which surround a hole, slot or other aperture in the shielding through which radiation may be administered, may be coated, covered with a film, or further treated. Such coatings, films, or treatments include one or more applications of a radiation reflective, refractive, diffractive, or scattering material that serves to focus or direct radiation away from the external shell or coupon of a shield and into the target site. Examples of such radiation reflective materials include glass, ceramic or metal foils or particles of these, as well as the application of nanostructured materials sized and shaped to reflect, refract, diffract or scatter photons of a particular wavelength or other particles used for radiation treatment. Two, three, four or more coatings or films may be applied to a shell or coupon, especially in areas adjacent to a target site when the shielding is fitted to the subject. The coatings or films may be the same or different, for example, coatings or films having different abilities to reflect, refract, diffract or scatter photons having different wavelengths may be layered on a shell or coupon. In some embodiments a coating will be painted or sprayed on a shell or coupon. In other embodiments a coating or film may be applied or embedded in the shell or coupon during 3D printing. Such coatings or films may range in thickness from 0.005, 0.01, 0.02, 0.05, 0.1, 0.2, 0.5, 1.0, 2.0 or 5.0 cm or any intermediate value within this range.

In other embodiments the portions of an external shell or coupon may be coated or otherwise covered with one or more radiation absorbing materials or shielding layers to provide a higher degree of protection from misdirected, scattered or leaked radiation around a target site. In a preferred embodiment, the radiation absorbing or shielding material is placed on or near a hole, slot or other aperture through which radiation passes to reach a target site for the purpose of reducing penetration of radiation outside of the target site.

A shielding layer applied to a shell or coupon may comprise discrete layers of one or more materials, such as a gold foil sheet or a polymer sheet applied to or embedded in a shell or coupon. In some embodiments, shielding materials comprise high Z materials, such as tantalum, gold, platinum, tin, steel, copper, aluminum, etc. (e.g., a 0.05 mm to 0.2 mm thickness metallic foil). A shielding layer may include one or more rods, braids, hollow rods, tubules (or tubes), bars, dots (or spheres), trapezoids, or other shapes. In some embodiments a coating will be painted or sprayed on a shell or coupon. In other embodiments a coating or film may be applied or embedded in the shell or coupon during 3D printing of a shell or coupon.

Such coatings or films may range in thickness from 0.005, 0.01, 0.02, 0.05, 0.1, 0.2, 0.5, 1.0, 2.0 or 5.0 cm or any intermediate value within this range.

The finite element method (FEM) or finite elements analysis is a powerful technique originally developed for numerical solution of complex problems in structural mechanics, and it remains the method of choice for complex systems. Using FEM, a structural system is modeled by a set of selected finite elements interconnected at discrete points called nodes. Elements may have physical properties such as thickness, coefficient of thermal expansion, density, Young's modulus, shear modulus and Poisson's ratio. Further description of this method is incorporated by reference to hypertext transfer protocol secure://en.wikipedia.org/wiki/Finite_element_method_in_structural_mechanics (last accessed Apr. 18, 2018).

Von Mises stress distribution. Von Mises stress is used to check whether a design will withstand a given load condition. Further description of Von Mises stress is incorporated by reference to hypertext transfer protocol secure://en.wikipedia.org/wiki/Von_Misesyield_criterion (last accessed Apr. 18, 2018); and to hypertext transfer protocol secure://en.wikipedia.org/wiki/Von_Mises_distribution (last accessed Apr. 18, 2018).

Virtual radiation shields. One embodiment of the invention is a virtual radiation shield and methods for using it to design a prototype or final radiation shield, to help plan a radiation treatment, or for radiological training. A virtual 3D radiation shield is generated by a computer software tool that achieves a desired patient fit and level of radiation blocking when filled with a particular amount of a radiation-blocking material. The virtual shield may be viewed and adjustments and refinements can be made to the dimensions or degree of shielding and checked against the patient's 3D images to achieve a final design for a prototype. Any suitable desired changes or refinement can be made at this point, such as, but not limited to, adjustments to shape, dimension, edges, size, smoothness, position, orientation, symmetry, and projection. After changes and refinements, the virtual shield can be re-situated in the original, untouched 3D image to test appropriateness, feasibility, and desirability of the refinements. Further adjustments with the morphing tools as above can be performed and the adjustments and refinements can be performed repeatedly until radiologist or other medical specialist is satisfied and an optimal implant is achieved for a particular radiological procedure. The virtual 3D shield and any other relevant information is saved on computer readable media preferably in the form of a CAD file. The file is then sent to a fabrication machine via wireless or wired communication. The data from the method of radiation shield creation can help prepare a radiologist for a subsequent radiological procedure on the patient. The final shield and 3D images can be used to create a virtual simulation of a surgical procedure to help position the shielding on a patient or position the patient for the procedure.. The shielding and 3D images may also be used to by a radiologist or medical student to virtually practice the radiological procedure.

### Example

The following non-limiting example illustrates aspects of one embodiment of the invention.

A specific radiation treatment plan for patient provides the clinical constraints for the engineering design, analysis, and manufacturing of the coupon and radiation shielding provided by the coupon once filled with a radiation-blocking material.

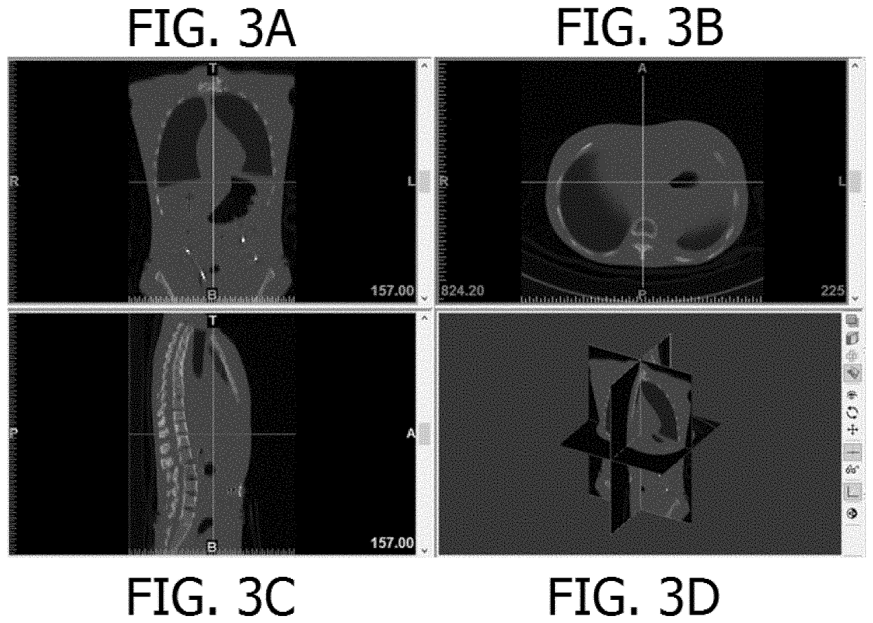

An example of a patient's CT scan and the detailed anatomy of the trunk is shown by FIG. 3.

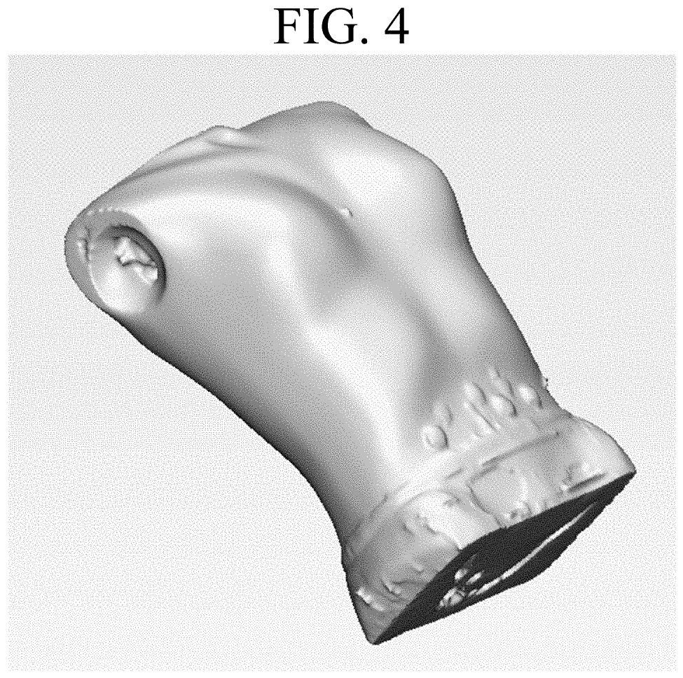

FIG. 4 shows an example of a 3D model of simplified patient's outline generated from CT scan. The same segmentation techniques can be applied to obtain specific tissue or organ's geometry for design purposes.

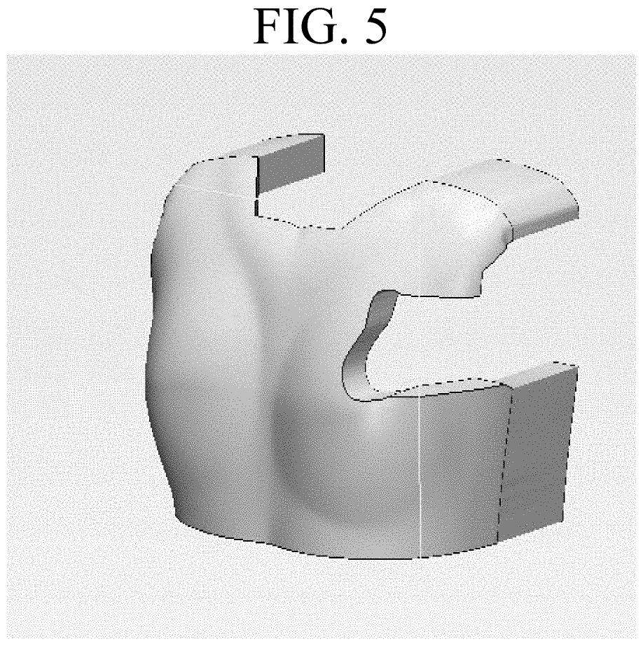

FIG. 5 shows an example of a radiation shield generated based on the patient's 3D model in FIG. 4. The shape of the shield can fit the patient leaving with calculated clearances to allow for releasing any pressure from the device on the patient without interfering with the functions of the device in shielding undesirable leakage and scatter radiation to non-target tissues.

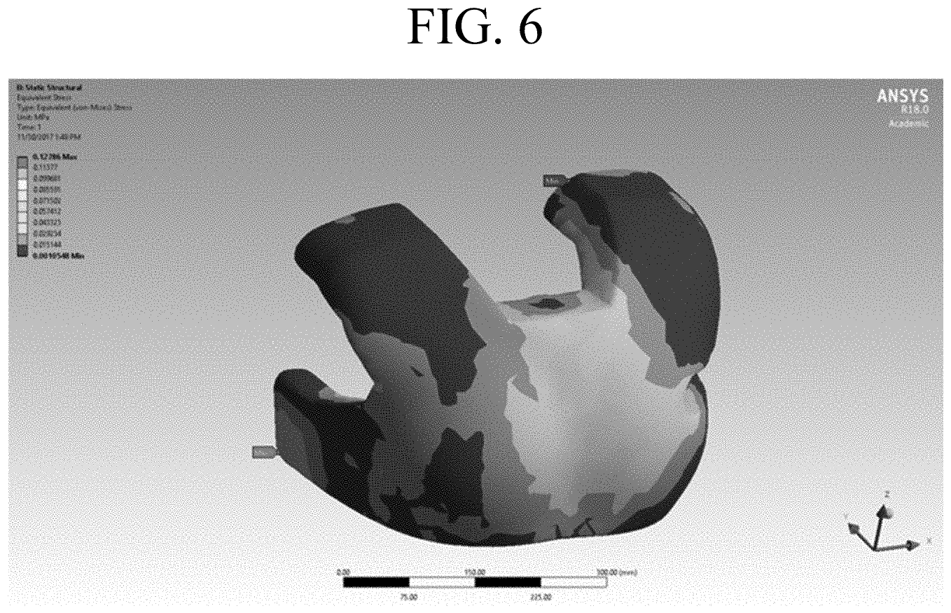

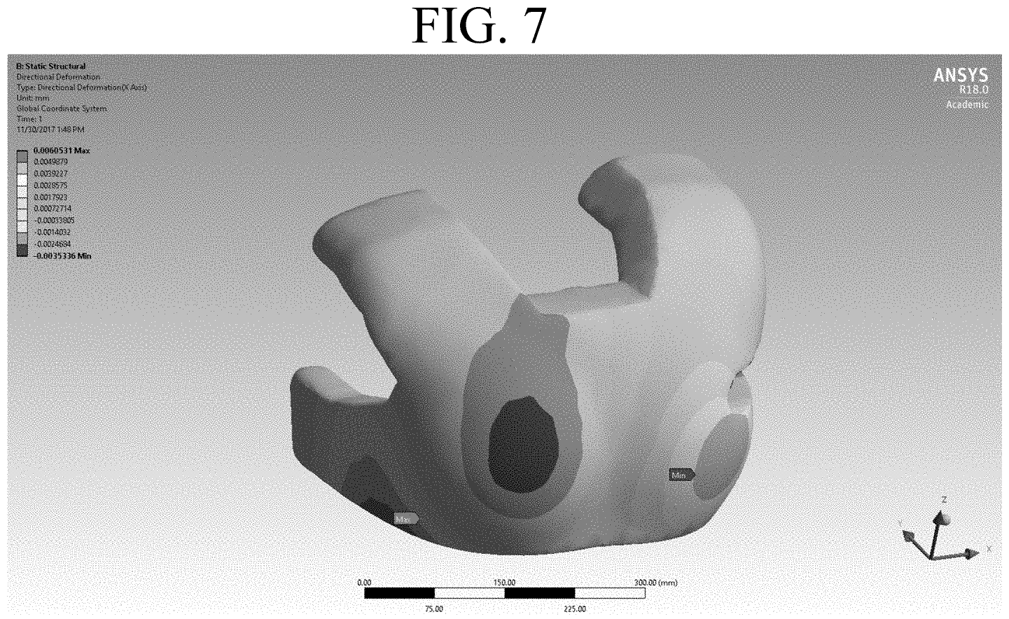

Finite element analysis is conducted on the developed shield design according to the introduced digital technology approach (DTA). The results reveal the distribution of stresses (FIG. 6) and deformation (FIG. 7) generated in the device due to its weight. A factor of safety of about 15 makes the developed device very safe with almost zero-potential failure during treatment. A factor of safety may be selected based on the nature of the radiological treatment and risk factors; for example, the factor of safety may be equal to or at least 2, 5, 10, 15, 20, or 30 (or any intermediate value within this range).

Iterations of design are conducted according to the DTA displayed in FIG. 2.

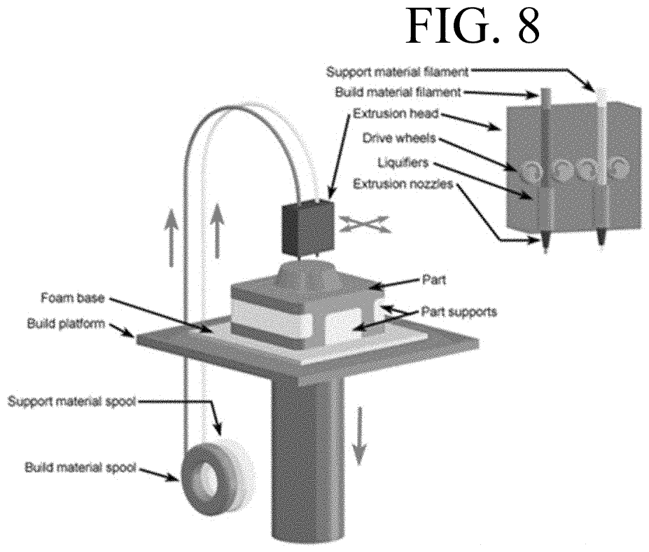

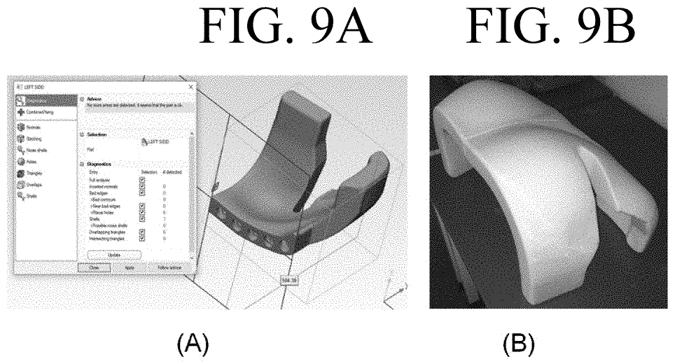

Once a suitable design of the novel device is realized, construction of the device is completed using additive manufacturing techniques (3D printing process). FIG. 8 displays schematics of 3D printing process used in building the tested prototype. FIG. 9 displays the developed split shield design in (9A) and the assembled shield in (9B).

FIGS. 6 to 9 respectively show Von Mises stress distribution due to the device's own weight (FIG. 6), deformation (distortion) distribution due to the device's own weight (FIG. 7), schematics of Fusion Deposition Modelling (3D Printing) process used in construction of the device (FIG. 8) and the developed split shield design is displayed in (FIG. 9A) and the assembled shield is shown in (FIG. 9B).

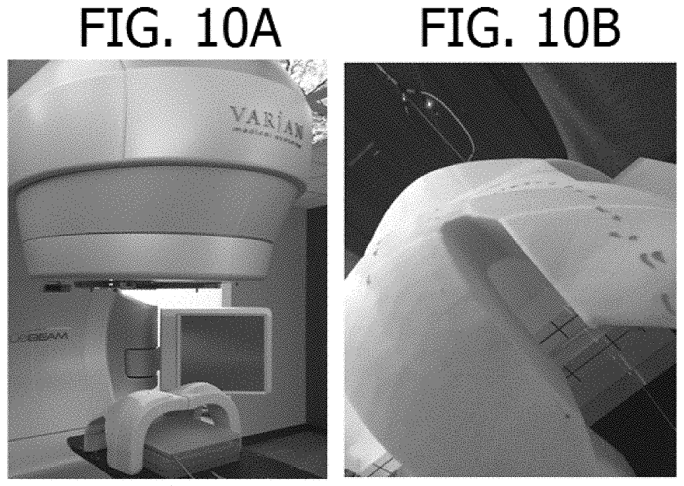

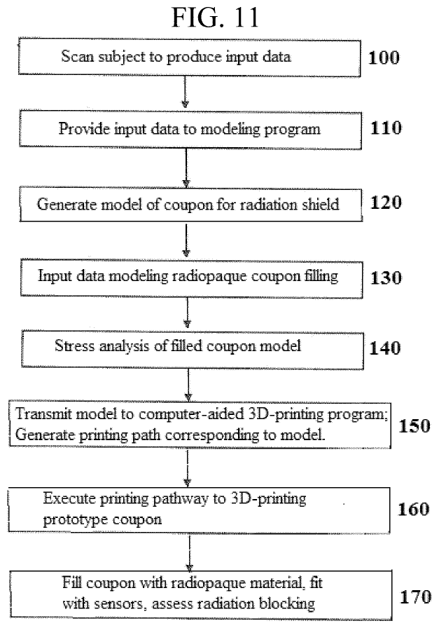

The final step in the development of the shielding device involves verifying its performance using full-scale prototype tested on a phantom mimicking the geometric attributes of the patient and equipped with several sensors for measuring the effectiveness of the novel design in reducing peripheral radiation dose during treating cancer patients. FIG. 10 displays the setup for testing the shield under LINAC machine. All measurements show that the developed TECHSHIELD is capable of reducing peripheral radiation dose affecting non target tissues by 50-60%, depending on direction of the radiation beam.

Although the invention herein has been described with reference to particular cases, it is to be under stood that these cases are merely illustrative of the principles and applications of the present invention. It is therefore to be understood that numerous modifications may be made to the illustrative cases and that other arrangements may be devised without departing from the scope of the present invention. For example, features and procedures described in relation to the illustrative case studies listed in the present claim might be modified in order to match the needs for other cases such as shields for protection of thyroids or other sensitive tissues other than chest area. In addition, although methods may be described as road map with number of steps listed in our novel Digital Technology Approach (DTA), the development procedures for new applications do not need to be followed in the same order listed in DTA.

Terminology used herein is for the purpose of describing particular embodiments only and is not intended to be limiting of the invention.

The headings (such as “Background” and “Summary”) and sub-headings used herein are intended only for general organization of topics within the present invention, and are not intended to limit the disclosure of the present invention or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

As used herein, the singular forms “a”, “an” and “the” are intended to include the plural forms as well, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, the term “and/or” includes any and all combinations of one or more of the associated listed items and may be abbreviated as “/”.

Links are disabled by spelling out of, or deletion of, http: or https: or by insertion of a space or underlined space before www. In some instances, the text available via the link on the “last accessed” date may be incorporated by reference.

As used herein in the specification and claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all subranges subsumed therein.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it also describes subranges for Parameter X including 1-9, 1-8, 1-7, 2-9, 2-8, 2-7, 3-9, 3-8, 3-7, 2-8, 3-7, 4-6, or 7-10, 8-10 or 9-10 as mere examples. A range encompasses its endpoints as well as values inside of an endpoint, for example, the range 0-5 includes 0, >0, 1, 2, 3, 4, <5 and 5.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology. As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Spatially relative terms, such as “under”, “below”, “lower”, “over”, “upper”, “in front of” or “behind” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

Numerous modifications and variations are possible in light of the above teachings. It is therefore to be understood that within the scope of the appended claims, the invention may be practiced otherwise than as specifically described herein.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

1. A method for making radiation shielding comprising:
(a) scanning a body or body part of a subject to produce scanning data that defines a target site to be treated with radiation and non-target sites to be protected;
(b) inputting the scanning data into a CAD program to produce a 3D CAD model of a hollow shell or coupon containing one or more cavities that can accommodate radiation shielding material and that is shaped so that when placed on the body of the subject exposes a target site to be irradiated and covers one or more non-target sites to be shielded from radiation;
(c) digitally assessing the 3D CAD model to determine distribution of stresses and deformations in the 3D CAD model of a hollow shell or coupon when filled with a radiation shielding material and selecting a 3D CAD model having a distribution of stresses and deformations that do not preclude its safe use on the subject during a radiation treatment,
(d) 3D printing or additively manufacturing (“AM”) a coupon or shell from the 3D CAD model which is filled with a radiation blocking material to produce a prototype radiation shielding from the 3D CAD model; and, optionally,
(e) testing the 3D-printed or additively manufactured prototype radiation shielding on a phantom subject equipped with one or more radiation sensors, and/or fitting the prototype radiation shielding on the subject and, optionally irradiating the subject with a test dosage of radiation, and
(f) further modifying the 3D CAD model based on results from testing the phantom subject or from results of testing the prototype on the subject and repeating (c) and (d) or repeating (d), or accepting the prototype model as the radiation shielding.

2. The method of claim 1, wherein (a) said scanning comprises making a CT/X-ray scan of the subject's body or body part.

3. The method of claim 1, wherein (a) comprises scanning a subject's neck, head, or portion thereof.

4. The method of claim 1, wherein (a) comprises scanning a subject's torso or portion thereof.

5. The method of claim 1, wherein (a) comprises scanning a subject's thorax or portion thereof.

6. The method of claim 1, wherein (a) comprises scanning a subject's abdomen or portion thereof.

7. The method of claim 1, wherein (a) comprises scanning a subject's arm or leg, or portion thereof.

8. The method of claim 1, wherein (a) said scanning comprises scanning a position of a tumor mass or tumor site to be exposed to radiation.

9. The method of claim 1, wherein (a) said scanning comprises locating a position of one or more organs, glands or tissues to be protected from radiation.

10. The method of claim 1, wherein (a) said scanning comprises locating a position of a fetus to be protected from radiation in a pregnant woman.

11. The method of claim 1, wherein (a) said scanning comprises locating a position of one or more pacemakers, prosthetics, or implanted devices to be shielded from radiation.

12. The method of claim 1, wherein (b) comprises producing a 3D CAD model that comprises two or more parts or subportions.

13. The method of claim 1, wherein (c) digitally assesses the 3D CAD model when filled with a radiation shielding that comprises bismuth, lead or tungsten, or mixtures thereof.

14. The method of claim 1, wherein (c) digitally assessing the 3D CAD model to determine distribution of stresses and deformations imposed by a weight of the radiation shielding when filled with a radiation shielding material comprises Von Mises stress analysis.

15. The method of claim 1, wherein (b) comprises producing a 3D CAD model that comprises two or more symmetrical subportions and (c) comprises digitally assessing each symmetrical subportion of the model to determine stress distribution.

16. The method of claim 1 that comprises (e).

17. The method of claim 16, wherein in (e) said sensors are gel dosimeters which measure radiation dosages in and around the target site in the phantom subject and/or in the subject receiving a test dose of radiation.

18. The method of claim 16, further comprising modifying the 3D CAD model after (c) digitally assessing the 3D CAD model to increase radiation exposure at the target site and/or reduce radiation exposure at non-target sites, and 3D or additively manufacturing radiation shielding from the modified 3D CAD model.

19. A 3D-printed or additively-manufactured radiation shielding that is made by the method of claim 1.

20. A method for treating a subject with radiation comprising covering non-target sites of the subject's body with the radiation shielding of claim 19, wherein said target site for radiation treatment is not covered.
